SEQUENCE LISTING (8 sequences)

SEQ ID NO: 1 - protein, 6 aa, synthetic construct
  REGION 1..6: note = Description of sequence: Unbound peptide
LVPRGS

SEQ ID NO: 2 - protein, 11 aa, synthetic construct
  REGION 1..11: note = Description of sequence: Unbound peptide
GGLVPRGSGAS

SEQ ID NO: 3 - protein, 13 aa, synthetic construct
  REGION 1..13: note = Description of sequence: Unbound peptide
AKGALVPRGSAGK

SEQ ID NO: 4 - protein, 11 aa, synthetic construct
  REGION 1..11: note = Description of sequence: Unbound peptide
  SITE 2: note = D-Phenylalanine
GFPRGFPAGGK

SEQ ID NO: 5 - protein, 6 aa, synthetic construct
  REGION 1..6: note = Description of sequence: Methacrylate bound peptide
  MOD_RES 1: note = Amino acid is bound to methacrylate cap
  MOD_RES 6: note = Amino acid is bound to methacrylate cap
LVPRGS

SEQ ID NO: 6 - protein, 11 aa, synthetic construct
  REGION 1..11: note = Description of sequence: Methacrylate bound peptide
  MOD_RES 1: note = Amino acid is bound to methacrylate cap
  MOD_RES 11: note = Amino acid is bound to methacrylate cap
GGLVPRGSGAS

SEQ ID NO: 7 - protein, 13 aa, synthetic construct
  REGION 1..13: note = Description of sequence: Methacrylate bound peptide
  MOD_RES 1: note = Amino acid is bound to methacrylate cap
  MOD_RES 13: note = Amino acid is bound to methacrylate cap
AKGALVPRGSAGK

SEQ ID NO: 8 - protein, 11 aa, synthetic construct
  REGION 1..11: note = Description of sequence: Methacrylate bound peptide
  SITE 2: note = D-Phenylalanine
  MOD_RES 1: note = Amino acid is bound to methacrylate cap
  MOD_RES 11: note = Amino acid is bound to methacrylate cap
GFPRGFPAGGK

(19) United States
(12) Patent Application Publication
(10) Pub. No.: US 2026/0306660 A1
(43) Pub. Date: Oct. 8, 2026

(54) THROMBIN SENSITIVE PLATELET LIKE PARTICLES

(71) Applicant: North Carolina State University, Raleigh, NC (US)

(72) Inventors: Ashley C. BROWN, Raleigh, NC (US); Kimberly A. NELLENBACH, Raleigh, NC (US); Eunice CHEE, Raleigh, NC (US); Emily MIHALKO, Raleigh, NC (US); Sanika PANDIT, Raleigh, NC (US); Krista HILMAS, Raleigh, NC (US)

(21) Appl. No.: 19/483,490

(22) PCT Filed: May 10, 2024
(86) PCT No.: PCT/US2024/028880

§ 371 Date: Nov. 11, 2025

Related U.S. Application Data
(60) Provisional application No. 63/501,533, filed on May 11, 2023.

(30) Foreign Application Priority Data
May 11, 2023 (US) 63/501533

(51) Int. Cl. A61K 47/69; A61K 47/68; A61L 26/00; A61P 17/02

(52) U.S. Cl. CPC A61K 47/6933; A61K 47/6843; A61L 26/0014; A61L 26/0066; A61P 17/02

(57) ABSTRACT

Disclosed herein are thrombin-sensitive platelet like particles. The particles can be used to promote healing in a variety of wounds and can be used in drug delivery systems.

### CROSS-REFERENCE TO RELATED APPLICATION

This application claims the benefit of U.S. Provisional Application 63/501,533, filed May 11, 2023, the contents of which are hereby incorporated in its entirety.

### ACKNOWLEDGEMENT OF GOVERNMENT SUPPORT

This invention was made with government support under Grant No. DMR1847488 awarded by the National Science Foundation. The government has certain rights in the invention.

### BACKGROUND

Under normal circumstances, platelets circulate in a round, inactive state in the circulatory system. In this state, platelets can easily marginate or move to the vessel wall for attachment. When injury occurs, platelets are prompted by injury-associated molecules, including thrombin, to shift into a stellate shape and increase surface exposure of fibrin-binding receptors. In this activated state, platelets can mediate the hemostatic process through platelet aggregation during primary hemostasis and promotion of fibrin formation during secondary hemostasis. After the formation of a fibrin-rich clot, platelets utilize actin-myosin machinery to pull on the fibrin network, resulting in clot retraction. Retracted clots have increased stiffness compared to unretracted clots, which enhances clot stability. Additionally, newer evidence suggests that increased stiffness due to clot retraction may contribute to healing after cessation of bleeding by guiding cells into the wound bed by durotaxis.

Synthetic platelet-like particles (PLPs) consisting of a microgel coupled to a fibrin-targeting antibody have been developed. Previous work in our group has demonstrated that a highly deformable microgel is mimetic of active platelet morphology and when combined with high fibrin targeting affinity, for example by coupling to a fibrin-targeting antibody, can recapitulate the clot retraction ability of natural platelets. As a result of this particle design, PLPs can hone specifically to injuries and enhance subsequent healing after cessation of bleeding.

The remains a need for improved platelet-like particles. There remains a need for improved particles that more closely mimic the functionality of naturally occurring platelets. There remains a need for improved methods and systems for treating injuries and wounds. There remains a need for improved methods and systems for treating traumatic brain injuries. There remains a need for improved methods and systems for delivering therapeutic agents, including chemotherapeutic agents to subjects in need thereof.

### BRIEF DESCRIPTION OF THE FIGURES

FIG. 1 depicts A) Schematic of survival liver laceration model B) Summary of blood loss data. Mice treated with TS-PLPs displayed significantly lower blood loss and bleeding area. Mice treated with TS-PLPs and size-matched ULC-PLPs displayed significant decreases in bleeding compared to control animals. n=6-8 animals per group. C) H&E stained wound sections with summary of wound area measurements. Scale bar=1000 μm. Mice treated with all hemostatic particles had significantly lower average wound area when compared to saline mice, though the standard deviation was lowest in the TS-PLP group. n=3-6 mice per group. *=p≤0.05, **=p≤0.01, ***=p≤0.001.

FIG. 2 depicts A) Schematic of TS-PLP Function. B) Representative cryoSEM image of TSNs prior to thrombin exposure. When unmodified, TSNs exhibit rounded morphology C) Representative AFM image and height trace of TSNs prior to thrombin exposure.

FIG. 3 depicts representative confocal image of unmodified TSN loaded with 70 kDa FITC labeled dextran (left). Fluorescent profile of 70 kDa loaded TSN indicates that there is lower signal in the middle of the particle compared to the edges, indicating that the 70 kDa dextran does not fully penetrate the particle. Since 70 kDa is close to the molecular size of thrombin, these observations may indicate that thrombin does not fully penetrate the nanogels, potentially explaining why full particle degradation is not observed.

FIG. 4 depicts A) Representative cryoSEM images of inactive platelets, activated platelets, and size-matched control 7% BIS crosslinked and ultralow crosslinked (ULC) microgels. Scale bar=1 μm B) Representative cryoSEM images of thrombin-sensitive nanogels (TSNs) at various time points following exposure to 75 U/mL thrombin.

FIG. 5 depicts representative cryoSEM images of fibrin clots polymerized in the presence of TS-PLPs, small ULC-PLPs, small 7 BIS-PLPs, and ULC-PLPs. Each image is 30 μm×30 μm. Scale bar=10 μm B) Mean percent porosity for each group of clots. Notably, significantly lower percent porosity was observed in small ULC-PLP and ULC-PLP groups in clots with no thrombin overlay but not in clots incorporated with unmodified TS-PLPs. After a thrombin overlay, significantly lower percent porosity was also observed in clots with TS-PLPs incorporated in addition to the ULC-PLP groups. n=9 images per group (3 images per clot, 3 clots per group). *=p≤0.05, **=p≤0.01, ***=p≤0.001.

FIG. 6 depicts a summary of terminal in vivo murine bleeding study results. A) Representative IHC images of wound tissue harvested immediately after injury and stained for fibrin and particles. Scale bar=100 μm B) Summary of total area of fibrin and particle signal, as quantified by ImageJ analysis. N=10-12 images/group. C) Total blood loss results from separate cohort of animals injected with saline or 5 mg/kg TS-PLPs. N=4 mice/group. *=p≤0.05.

FIG. 7 depicts A) Representative IHC wound sections collected 1 week after injury where cell nuclei, fibrin and particles were fluorescently labeled (top row) and cell nuclei and platelets were fluorescently labeled (bottom row). Scale bar=400 μm B) Summary of total area of fibrin, particles, and platelet presence (CD61+). Mice treated with TS-PLPs were observed to have both significantly higher fibrin presence while there were no significant differences observed in platelet presence and particle presence. n=3 images per wound and 3-5 animals per group. *=p≤0.05, ***=p≤0.001.

FIG. 8 depicts a summary of off-target effects studies. A) Representative images of both H&E to characterize gross tissue morphology and fluorescently labeled peripheral organs for fibrin and particle presence. Images are 400 μm×400 μm. Scale bar=100 μm B) Summary of total area for fibrin and particle presence, separated by organ type. Fibrin and particle presence were analyzed by particle analysis in ImageJ. For each organ type, n=3-5 animals were analyzed per group. Overall, no significant differences in both fibrin and particle presence were observed with relatively low total area detected (compared to total area detected in wounds).

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein, “(meth)acrylic acid” covers both acrylic acid and methacrylic acid, alone or in combination. The same convention is employed for (meth)acrylate and (meth)acrylamide.

As used herein a “biologic drug” is a macromolecular biopolymer, e.g., a protein (including antibodies), a polysaccharide, or a nucleic acid. Biological drugs are listable in the FDA Purple Book. A “small molecule drug” is a therapeutic agent typically having a molecular weight of 1,500 Da or less. Small molecule drugs are listable in the FDA Orange Book.

Compounds disclosed herein may be provided in the form of physiologically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are shape-changing platelet-like particles. The particles change shape when contacted with thrombin, thereby mimicking the behavior of naturally occurring platelets. The particles may be designated Thrombin-Sensitive Platelet Like Particles (“TS-PLPs”). In certain implementations, the TS-PLPs may be provided as a nanogel, i.e., a thrombin-sensitive nanogel. In certain implementations, the TS-PLPs include a polymer crosslinked with thrombin degradable crosslinkers. In some implementations, the TS-PLPs include a poly(meth)acrylamide crosslinked with thrombin degradable crosslinkers, i.e., a thrombin-degradable crosslinked poly(meth)acrylamide.

In some implementations the TS-PLPs can be core/shell particles. In some implementations the TS-PLPs are core/shell particles having a core including a poly(meth)acrylamide crosslinked with a thrombin degradable crosslinker. In some implementations the TS-PLPs are core/shell particles having a shell including a poly(meth)acrylamide crosslinked with a thrombin degradable crosslinker. In some implementations the TS-PLPs are core/shell particles in which both the core and the shell include a poly(meth)acrylamide crosslinked with a thrombin degradable crosslinker. In some implementations the TS-PLPs are core/shell particles having a core including poly(meth)acrylamide crosslinked with a thrombin degradable crosslinker, and a shell including a crosslinked polymer, for example a crosslinked poly(meth)acrylamide, that is not thrombin degradable.

In some implementations, the TS-PLPs can include a crosslinked polyNHC₁₋₆alkyl(meth)acrylamide, a polyN(di-C₁₋₆alkyl)(meth)acrylamide, or a combination thereof. In certain implementations, the TS-PLPs can include a crosslinked poly(N-isopropyl)acrylamide.

In certain implementations, the crosslinked poly(meth)acrylamide is a monopolymer, while in other implementations the crosslinked poly(meth)acrylamide can be a co-polymer. In certain implementations, the TS-PLPs include a crosslinked poly [(meth)acrylamide-co-acrylic acid] copolymer. In certain implementations, the TS-PLPs include a crosslinked poly(N-isopropylacrylamide-co-acrylic acid) copolymer.

Thrombin-degradable crosslinkers include chemical moieties that are cleaved in the presence of thrombin. In certain implementations the thrombin degradable crosslinker includes a 5-residue peptidyl fragment having the formula -Pro-Arg-X¹-X²-Arg-, wherein X¹ can be Ser, Ala, Gly, or Thr, and X² is a non-acidic amino acid, for example Ala, Alle, Arg, Asn, Cys, Gln, Gly, His, Hyp (hydroxyproline), Ile, Leu, Lys, Met, NaI (naphthyl alanine), Phe, Pro, Sec, Ser, Thr, Tyr, Trp, Val, or Phg (phenylglycine). In certain implementations, X² can be Ala, Alle, Cys, Gly, Hyp, Ile, Leu, Met, NaI, Phe, Pro, Ser, Thr, Tyr, Trp, Val, or Phg (phenylglycine). The crosslinker may include additional amino acid residues, so long at the 5-residue peptide above is present.

In certain implementations, the thrombin degradable crosslinker includes the sequence:

[SEQ ID NO: 1]
Leu-Val-Pro-Arg-Gly-Ser;
[SEQ ID NO: 2]
Gly-Gly-Leu-Val-Pro-Arg-Gly-Ser-Gly-Ala Ser;
[SEQ ID NO: 3]
Ala-Lys-Gly-Ala-Leu-Val-Pro-Arg-Gly-Ser-Ala-Gly-
Lys;
or
[SEQ ID NO: 4]
Gly-dPHe-Pro-Arg-Gly-Phe-Pro-Ala-Gly-Gly-Lys.

The N-terminus and C-terminus of the thrombin degradable crosslinker may be functionalized with (meth)acrylate residues in order to integrate into the poly(meth)acrylamide network during monomer polymerization.

The TS-PLPs can have thrombin-degradable crosslinking density (i.e., the crosslinking density of the thrombin-degradable crosslinks) that is no greater than 20%, no greater than 15%, no greater than 12.5%, no greater than 10%, no greater than 7.5%, no greater than 5%, no greater than 2.5%, no greater than 2.0%, no greater than 1.5%, no greater than 1.0%, no greater than 0.75%, no greater than 0.50%, no greater than 0.25%, no greater than 0.10%, or no greater than 0.05%. In certain implementations, the crosslinking density is calculated based on the ratios of the monomers used to prepare the crosslinked poly(meth)acrylamide. When a particle is described to have a thrombin-degradable crosslinking density no greater than a given value, the particle does have at least some thrombin-degradable crosslinks. In other words, a particle with no thrombin-degradable crosslinks is not a particle having a thrombin-degradable crosslinking density less than, e.g., 5%.

In certain implementations, the TS-PLPs can have thrombin-degradable crosslinking density (i.e., the crosslinking density of the thrombin-degradable crosslinks) that from 15% to 0.5%, from 15% to 2.5%, from 15% to 5%, from 15% to 10%, from 12.5% to 7.5%, from 10% to 5%, from 7.5% to 2.5%, from 5% to 1%, from 2.5% to 1%. In certain implementations, the crosslinking density is calculated based on the wt. ratios of the monomers used to prepare the crosslinked poly(meth)acrylamide.

The TS-PLPs can be characterized by polydispersity. In some implementations, the TS-PLPs have a polydispersity index no greater than 0.4, no greater than 0.3, no greater than 0.25, no greater than 0.2, no greater than 0.15, or no greater than 0.1, wherein the polydispersity index is calculated from the square of the standard deviation divided by the mean particle diameter determined from Nanosight analysis.

The TS-PLPs may further include one or more therapeutic agents. In some implementations, the TS-PLPs can include a fibrin binding moiety. In some implementations, the fibrin binding moiety is covalently conjugated to the TS-PLPs. The fibrin binding moiety can be covalently conjugated to (meth)acrylic acid residues in the crosslinked polymer. In certain implementations, the TS-PLPs are core/shell particles and the fibrin binding moiety is covalently conjugated to the shell polymer.

In certain implementations, the fibrin binding moiety includes one or more fibrin-binding IgG antibody, fibrin-binding peptide (i.e. fibrin knob mimic), Fragment D binding antibody; or other antibody fragments (sdFvs, etc.) that bind fibrin.

In some implementations, the TS-PLPs can include one or more antibiotics, antivirals, anti-inflammatoirenalgesics, anti-coagulants, fibrinolytics, chemotherapeutics, or growth factors. In some implementations, the TS-PLPs can include a small molecule drug, while in other implementations the TS-PLPs can include a biologic drug. In some implementations the TS-PLPs can include both a small molecule drug (or drugs) and a biologic drug.

In some implementations, the TS-PLPs can include an anti-fibrin antibody.

The TS-PLPs can be characterized by their dry diameter. In certain implementations, the TS-PLPs can have an average dry diameter no greater than 1,000 nm, no greater than 750 nm, no greater than 500 nm, no greater than 300 nm, no greater than 275 nm, no greater than 250 nm, no greater than 225 nm, or no greater than 200 nm.

In certain implementations, the TS-PLPs can have an average dry height no greater than 50 nm, no greater than 28 nm, no greater than 25 nm, or no greater than 20 nm.

In some implementations, the crosslinked poly(meth)acrylamide is derived from a mixture of monomers, wherein the monomer mixture includes a (meth)acrylamide monomer and a thrombin-degradable crosslinking monomer. In certain implementations the thrombin-degradable crosslinking monomer includes a 5-residue peptidyl fragment having the formula-Pro-Arg-X¹-X²-Arg-, wherein X¹ and X² are as defined above. The crosslinking monomer may include additional amino acid residues, so long at the 5-residue fragment above is present.

In certain implementations, the thrombin degradable crosslinking monomer includes the compound:

[SEQ ID NO: 5]
methacrylate-Leu-Val-Pro-Arg-Gly-Ser-
methacrylate;
[SEQ ID NO: 6]
methacrylate-Gly-Gly-Leu-Val-Pro-Arg-Gly-Ser-
Gly-Ala Ser-methacrylate;
[SEQ ID NO: 7]
methacrylate-Ala-Lys-Gly-Ala-Leu-Val-Pro-Arg-
Gly-Ser-Ala-Gly-Lys-methacrylate;
or
[SEQ ID NO: 8]
methacrylate-Gly-dPHe-Pro-Arg-Gly-Phe-Pro-Ala-
Gly-Gly-Lys-methacrylate.

In some implementations, all the crosslinking monomers in the monomer mixture are thrombin-degradable crosslinking monomers, i.e., the monomer mixture does not include a non-thrombin degradable crosslinker.

The (meth)acrylamide monomer can be a NHC₁₋₆alkyl(meth)acrylamide, a N(di-C₁₋₆ alkyl)(meth)acrylamide, or a combination thereof. In certain implementations the (meth)acrylamide monomer can be a NHC₁₋₆alkylacrylamide, a N(di-C₁₋₆alkyl) acrylamide, or a combination thereof. In some implementations, the (meth)acrylamide monomer includes N-methylacrylamide, N-ethylacrylamide, N-propylacrylamide, N-isopropylacrylamide, N-butylacrylamide, N-isobutylacrylamide, N-sec-butylacrylamide, N-tert-butylacrylamide, N,N-dimethylacrylamide, N,N-diethylacrylamide, N,N-dipropylacrylamide, N,N-di-isopropylacrylamide, N-methyl-N-ethylacrylamide, N-methyl-N-propylacrylamide, N-ethyl-N-propylacrylamide, N-methyl-N-isopropylacrylamide, N-ethyl-N-isopropylacrylamide, or a combination thereof.

In certain implementations, the (meth)acrylamide monomer is N-isopropylacrylamide.

In certain implementations, N-isopropylacrylamide is the only (meth)acrylamide present in the monomer mixture. In certain implementations, N-isopropylacrylamide and the thrombin-degradable crosslinking monomer are the only monomers in the monomer mixture.

In certain implementations, the monomer mixture may include other monomers, for example (meth)acrylic acid monomer. In some implementations, the monomer mixture includes acrylic acid.

In certain implementations, N-isopropylacrylamide, the thrombin-degradable crosslinking monomer, and acrylic acid are the only monomers in the monomer mixture.

In some implementations, the monomer mixture includes 65-90 wt. % (meth)acrylamide monomers, 2-20 wt. % thrombin-degradable crosslinking monomers, and 0-15 wt. % (meth)acrylic acid monomers. In certain implementations, the monomer mixture includes 80-90 wt. % (meth)acrylamide monomers, 2.5-7.5 wt. % thrombin-degradable crosslinking monomers, and 2.5-17.5 wt. % (meth)acrylic acid monomers. In some implementations, the monomer mixture includes about 85% (meth)acrylamide monomers, about 5% thrombin degradable monomers, and about 10% (meth)acrylic acid monomers.

The monomer mixture can also include a solvent, and in certain implementations, the monomer mixture includes water. In certain implementations, the total concentration of all monomers in the mixture can be from 0.5-1,000 mM, from 1-500 mM, from 10-500 mM, from 25-250 mM, from 25-150 mM, from 10-50 mM, from 25-75 mM, from 50-100 mM, from 75-125 mM, from 100-150 mM, from 125-175 mM, or from 150-200 mM.

In certain implementations, the monomer mixture can include an initiator. When present, the initiator can be present at a concentration from 0.001-500 mM, from 0.001-10 mM, from 0.001-1 mM, from 0.001-0.1 mM, from 0.001-0.01 mM, from 0.001-0.005 mM, from 0.005-0.01 mM, from 0.01-0.05 mM, from 0.05-0.1 mM, from 0.1-1 mM, from 1-500 mM, from 1-250 mM, from 10-250 mM, from 25-200 mM, from 50-150 mM, from 25-75 mM, from 50-100 mM, from 75-125 mM, from 100-150 mM, from 125-175 mM, or from 150-200 mM.

In some implementations, the initiator includes a persulfate, AIBN, ABCN, AAPH, or a combination thereof. In certain implementations, the initiator includes ammonium persulfate and tetramethylethylenediamine.

In some implementations, the initiator includes 50-150 mM ammonium persulfate and 0-125 mM tetramethylethylenediamine, preferably 100-150 mM ammonium persulfate and 75-125 mM tetramethylethylenediamine, more preferably 110-150 mM ammonium persulfate and 75-100 mM tetramethylethylenediamine.

In some implementations the initiator includes 0.001-0.1 mM ammonium persulfate and 0-0.1 mM tetramethylethylenediamine, preferably 0.01-0.1 mM ammonium persulfate and 0.005-0.05 mM tetramethylethylenediamine, more preferably 0.01-0.05 mM ammonium persulfate and 0.005-0.015 mM tetramethylethylenediamine.

The monomer mixture may be stirred to produce the TS-PLPs. In certain implementations, the mixture is stirred at a rate of 300-600 rpm, from 300-500, from 400-600, from 300-450, from 350-500, from 400-550, or from 450-600. At the time the initiator is added to the monomer mixture, the oxygen content of the monomer mixture can be less than 2 vol. %, less than 1 vol. %, less than 0.5 vol. %, less than 0.1 vol. %, less than 0.05 vol. %, or less than 0.01 vol. %.

Prior to the addition of the initiator, the monomer mixture may be stirred for a period of at least 10 minutes, at least 20 minutes, at least 30 minutes, or at least 60 minutes prior to the addition of the initiator. In some implementations, a (meth)acrylamide monomer, (meth)acrylic acid monomer, and thrombin-degradable crosslinker monomer are stirred for a period of at least 10 minutes, at least 20 minutes, at least 30 minutes, or at least 60 minutes prior to the addition of the initiator.

Also disclosed herein are methods of making thrombin-degradable crosslinked poly(meth)acrylamides including the step of polymerizing a mixture comprising a (meth)acrylamide and a thrombin-degradable crosslinker. The mixture may further include a (meth)acrylic acid, e.g., acrylic acid, as described herein.

In some implementations, the TS-PLPs are prepared by polymerizing a mixture including a NHC₁₋₆alkyl(meth)acrylamide, a N(di-C₁₋₆alkyl)(meth)acrylamide, or a combination thereof.

In some implementations, the TS-PLPs are prepared by polymerizing a mixture including a NHC₁₋₆alkylacrylamide, a N(di-C₁₋₆alkyl) acrylamide, or a combination thereof.

In some implementations, the TS-PLPs are prepared by polymerizing a mixture including N-methylacrylamide, N-ethylacrylamide, N-propylacrylamide, N-isopropylacrylamide, N-butylacrylamide, N-isobutylacrylamide, N-sec-butylacrylamide, N-tert-butylacrylamide, N,N-dimethylacrylamide, N,N-diethylacrylamide, N,N-dipropylacrylamide, N,N-di-isopropylacrylamide, N-methyl-N-ethylacrylamide, N-methyl-N-propylacrylamide, N-ethyl-N-propylacrylamide, N-methyl-N-isopropylacrylamide, N-ethyl-N-isopropylacrylamide, or a combination thereof.

In some implementations, the TS-PLPs are prepared by polymerizing a mixture including N-isopropylacrylamide.

The TS-PLPs can be prepared by polymerizing a mixture including a thrombin-degradable crosslinker that includes the 5-residue peptidyl fragment having the formula-Pro-Arg-X¹-X²-Arg-, wherein X¹ and X² are as defined above.

In certain implementations, the thrombin degradable crosslinker includes the compound:

[SEQ ID NO: 5]
methacrylate-Leu-Val-Pro-Arg-Gly-Ser-
methacrylate;
[SEQ ID NO: 6]
methacrylate-Gly-Gly-Leu-Val-Pro-Arg-Gly-Ser-
Gly-Ala Ser-methacrylate;
[SEQ ID NO: 7]
methacrylate-Ala-Lys-Gly-Ala-Leu-Val-Pro-Arg-
Gly-Ser-Ala-Gly-Lys-methacrylate;
or
[SEQ ID NO: 8]
methacrylate-Gly-dPHe-Pro-Arg-Gly-Phe-Pro-Ala-
Gly-Gly-Lys-methacrylate.

Also disclosed herein are methods of making thrombin-degradable crosslinked poly(meth)acrylamides including the step of polymerizing a mixture comprising a (meth)acrylamide and a thrombin-degradable crosslinker, wherein the mixture does not include a non-thrombin degradable crosslinker. In certain implementations, the polymerization reaction is a precipitation polymerization reaction.

In some implementations, the TS-PLPs are prepared by polymerizing a mixture including N-isopropylacrylamide, acrylic acid, and a thrombin-degradable crosslinking monomer. In certain implementations, no other reactive monomer compounds (i.e., compounds with (meth)acryl groups) are present.

In some implementations, the TS-PLPs are prepared by polymerizing a mixture including 65-90 wt. % (meth)acrylamide monomers, 2-20 wt. % thrombin-degradable crosslinking monomers, and 0-15 wt. % (meth)acrylic acid monomers. In certain implementations, the monomer mixture includes 80-90 wt. % (meth)acrylamide monomers, 2.5-7.5 wt. % thrombin-degradable crosslinking monomers, and 2.5-17.5 wt. % (meth)acrylic acid monomers. In some implementations, the monomer mixture includes about 85% (meth)acrylamide monomers, about 5% thrombin degradable monomers, and about 10% (meth)acrylic acid monomers.

The monomers may be provided in a solvent, for example water, when conducting the polymerization reaction. In some implementations, the total concentration of all monomers in the mixture can be from 0.5-1,000 mM, from 1-500 mM, from 10-500 mM, from 25-250 mM, from 25-150 mM, from 10-50 mM, from 25-75 mM, from 50-100 mM, from 75-125 mM, from 100-150 mM, from 125-175 mM, or from 150-200 mM.

In certain implementations, the polymerization can be conducted in the presence of an initiator. When present, the initiator can be present at a concentration from 0.001-500 mM, from 0.001-10 mM, from 0.001-1 mM, from 0.001-0.1 mM, from 0.001-0.01 mM, from 0.001-0.005 mM, from 0.005-0.01 mM, from 0.01-0.05 mM, from 0.05-0.1 mM, from 0.1-1 mM, from 1-500 mM, from 1-250 mM, from 10-250 mM, from 25-200 mM, from 50-150 mM, from 25-75 mM, from 50-100 mM, from 75-125 mM, from 100-150 mM, from 125-175 mM, or from 150-200 mM.

In some implementations, the initiator includes a persulfate, AIBN, ABCN, AAPH, or a combination thereof. In certain implementations, the initiator includes ammonium persulfate and tetramethylethylenediamine.

In some implementations, the initiator includes 50-150 mM ammonium persulfate and 0-125 mM tetramethylethylenediamine, preferably 100-150 mM ammonium persulfate and 75-125 mM tetramethylethylenediamine, more preferably 110-150 mM ammonium persulfate and 75-100 mM tetramethylethylenediamine.

In some implementations the initiator includes 0.001-0.1 mM ammonium persulfate and 0-0.1 mM tetramethylethylenediamine, preferably 0.01-0.1 mM ammonium persulfate and 0.005-0.05 mM tetramethylethylenediamine, more preferably 0.01-0.05 mM ammonium persulfate and 0.005-0.015 mM tetramethylethylenediamine.

The mixture may be stirred while conducting the polymerization reaction. In some implementations, prior to the addition of the initiator, some or all of the monomer components may be stirred at a rate of 300-600 rpm, from 300-500, from 400-600, from 300-450, from 350-500, from 400-550, or from 450-600. At the time the initiator is added to the monomer mixture, the oxygen content of the mixture can be less than 2 vol. %, less than 1 vol. %, less than 0.5 vol. %, less than 0.1 vol. %, less than 0.05 vol. %, or less than 0.01 vol. %.

In some implementations, the monomer mixture may be stirred for a period of at least 10 minutes, at least 20 minutes, at least 30 minutes, or at least 60 minutes prior to the addition of the initiator. In some implementations, a (meth)acrylamide monomer and thrombin-degradable crosslinker monomer are stirred for a period of at least 10 minutes, at least 20 minutes, at least 30 minutes, or at least 60 minutes prior to the addition of the initiator.

In some implementations, the (meth)acrylamide monomer and thrombin-degradable crosslinker monomer are stirred for a period of at least 10 minutes, at least 20 minutes, at least 30 minutes, or at least 60 minutes, followed by addition of a (meth)acrylic acid monomer. In some implementations, a (meth)acrylamide monomer, (meth)acrylic acid monomer, and thrombin-degradable crosslinker monomer are stirred for a period of at least 10 minutes, at least 20 minutes, at least 30 minutes, or at least 60 minutes prior to the addition of the initiator.

In some implementations, the monomer mixture may be stirred for a period of 1-48 hours, of 6-28 hours, of 12-48 hours, of 24-48 hours, of 36-48 hours, of 24-36 hours, of 12-36 hours, or of 12-24 hours following addition of the initiator.

The crosslinked particles may be separated from the reaction mixture and optionally purified, for instance by dialysis.

In some implementations, the separated crosslinked particles may be conjugated to a fibrin binding moiety, for instance using standard EDC/NHS chemistry.

In some implementations, the separated crosslinked particles may be combined in a second monomer mixture, which is then polymerized to produce core/shell TS-PLPs. In some implementations, the second monomer mixture can include a (meth)acrylamide monomer as defined herein, for example N-isopropylacrylamide. In some implementations, the second monomer mixture can include a (meth)acrylic acid, for example acrylic acid.

In some implementations, the second monomer mixture can include one or more crosslinking monomers. In some implementations, the second monomer mixture can include a thrombin-degradable crosslinking monomer, as defined herein. In some implementations, the second monomer mixture can include a crosslinking monomer that is not degraded by thrombin. Exemplary non-thrombin degradable crosslinking monomers include N,N′-methylenebis(acrylamide), N,N-(1,2-dihydroxyethylene)bisacrylamide, ethylene glycol diacrylate, di(ethylene gycol) diacrylate, tetra(ethylene glycol) diacrylate, ethylene glycol dimethacrylate, di(ethylene glycol) dimethacrylate, tri(ethylene glycol) dimethacrylate, or a combination thereof.

In some implementations, the second monomer mixture can include N-isopropylacrylamide, acrylic acid, and a thrombin-degradable crosslinking monomer.

In some implementations, the second monomer mixture can include N-isopropylacrylamide, acrylic acid, and a non-thrombin-degradable crosslinking monomer.

In certain implementations, the second monomer mixture comprises 85-100% N-isopropylacrylamide, 0-10% acrylic acid, and 0-10% non-thrombin-degradable crosslinking monomer. In some implementations, the second monomer mixture includes 90-95% N-isopropylacrylamide, 2.5-7.5% acrylic acid, and 1-2.5% non-thrombin-degradable crosslinking monomer.

After polymerizing the second monomer mixture, the core/shell TS-PLPs may be separated from the reaction mixture, and optionally purified for example by dialysis. The separated core/shell particles may be conjugated to a fibrin binding moiety.

Therapeutic agents may be loaded in the TS-PLPs by combining the TS-PLPs and agent(s) in a solution together. Alternatively, or additionally, an agent may be present the monomer mixtures described above. The small molecule therapeutic may be present at a concentration from 1-10,000 μM, from 10-10,000 μm, from 50-5,000 μM, from 100-5,000 μm, from 100-2,500 μM, from 100-1,000 μm, or from 250-1,000 μM. In other implementations, the therapeutic agent may be conjugated to the TS-PLPs, for instance covalently conjugated to the particle via (meth)acrylic residues. When the TS-PLP is a core/shell particle, the therapeutic agent may be conjugated to the core crosslinked polymer, to the shell crosslinked polymer, or to both the core crosslinked polymer and shell crosslinked polymer.

Also disclosed herein are pharmaceutical compositions including TS-PLPs. In certain implementations, the pharmaceutical composition is a solution suitable for parenteral or oral administration. In some implementations the pharmaceutical composition is a lyophilized powder, optionally including one or more solid excipients. Lyophilized powders can include water in an amount no greater than 5 wt. %, no greater than 2.5 wt. %, no greater than 1 wt. %, no greater than 0.5 wt. %, or no greater than 0.1 wt. %. The lyophilized composition may then be reconstituted in water prior to administration to a subject in need.

In certain implementations, the TS-PLPs may be combined with water to provide a thrombin-sensitive nanogel. The thrombin-sensitive nanogel can include TS-PLPs in an amount from 1-100 mg/ml, from 1-50 mg/ml, from 1-5 mg/ml, from 1-10 mg/ml, from 5-10 mg/ml, from 10-50 mg/ml, from 10-25 mg/ml, from 25-50 mg mg/ml, from 25-75 mg/ml, from 50-100 mg/ml, from 50-75 mg/ml, or from 75-90 mg/ml.

In some implementations, the TS-PLPs are provided in a topical formulation. Suitable formulations include ointments, pastes, creams, lotions, gels, powders, solutions, sprays, inhalants and/or patches. Generally, the TS-PLPs are admixed under sterile conditions with a pharmaceutically acceptable excipient and/or any needed preservatives and/or buffers as may be required. Additionally, the present invention contemplates the use of transdermal patches, which often have the added advantage of providing controlled delivery of TS-PLPs to the body. Such dosage forms may be prepared, for example, by dissolving and/or dispensing the particles in the proper medium. Alternatively, or additionally, the rate may be controlled by either providing a rate controlling membrane and/or by dispersing the active ingredient in a polymer matrix and/or gel.

The TS-PLPs may be administered to a subject in a variety of ways. In some implementations, the particles may be administered parenterally for example intravenously, intrathecally, intramuscularly, or subcutaneously. In some implementations the particles may be administered topically.

In some implementations, the TS-PLPs are used to promote wound healing. Exemplary wounds that may be treated with TS-PLPs include a trauma wound, a surgical wound, a burn wound, or an ulcer wound. Wound patients with coagulation disorders may be advantageously treated with TS-PLPs, for instance, diabetics, hemophiliacs, patients with vitamin K deficiency, Von Willebrand disease or other clotting factor deficiencies. The TS-PLPs can be used to treat wounds in patients undergoing anti-coagulation therapy, for instance patients receiving heparin, fandaparinux, idraparinux, vitamin K, coumadin, direct thrombin inhibitors like argatroban, dabigatran, factor Xa inhibitors like rivaroxaban, apixaban and edoxaban, anti-platelet agents such as clopidogrel and prasugrel. The TS-PLPs can be used to treat wounds arising from medical procedures such as percutaneous coronary intervention, stent and/or valve placement or repair, transfusion, and dialysis.

In some implementations, the trauma wound is a traumatic brain injury (“TBI”). The TS-PLPs may be administered, for example intravenously, at a dose level of 0.1-10 mg/kg. In certain implementations, the crosslinked particles are administered to the subject a single time, while in some implementations, the subject receives TS-PLPs more than one separate dosing event.

In some implementations, the ulcer wound is a decubitus ulcer, diabetic ulcer, gastrointestinal ulcer, or a combination thereof.

In some implementations, the wound is an infected wound, for example the wound is infected with one or more of a bacterial infection, a viral infection, a fungal infection, a parasitic infection. In certain implementations, the TS-PLPs may be administered in combination with one or more anti-infective agents. Such agents may be co-formulated with TS-PLPs for single dose administration or they may be administered separately.

Depending on the specific clinical indication, the TS-PLPs may be administered in combination with one or more additional active agents. In certain implementations, the TS-PLPs are administered in combination with an anti-clotting agent, for example anti-thrombin III, fibrinolytic drugs (tPA, plasminogen, TXA). In some implementations, the TS-PLPs are administered in combination with pro-clotting agents, for example blood components, plasma, platelets, recombinant clotting factors, pro-thrombin complex concentrate. Such agents may be co-formulated with the TS-PLPs or they may be administered separately.

In certain implementations, the TS-PLPs can be used as a component in a drug-delivery system. Therapeutic agents may be combined with TS-PLPs and selectively directed to desired locations within the body. In some implementations, the TS-PLPs are combined with one or more chemotherapeutic agents.

### Examples

While stirred at 450 rpm, 85% NIPMAM and 5% methacrylate-Gly-dPHe-Pro-Arg-Gly-Phe-Pro-Ala-Gly-Gly-Lys-methacrylate [SEQ ID NO: 8] were incubated in 95 mM TEMED and put under nitrogen purge for 1 hr at room temperature. 50 minutes into the purge, 10% Acrylic Acid was added. The reaction was then initiated at room temperature with 125 mM ammonium persulfate (APS) and allowed to react for 24 hours. The resulting reaction mixture was then purified by dialysis in ultrapure water in 1000 kDa dialysis tubing for 72 hours with water changes every 24 hours. The particle solution was then lyophilized and resuspended at desired concentrations with ultrapure water to give thrombin-sensitive nanogels, and stored at 4° C.

To control for particle size effects, control microgels, size-matched to thrombin-sensitive nanogels, were synthesized. Ultralow crosslinked microgels (ULCs) and 7% crosslinked BIS (7% BIS) were selected as highly deformable and nondeformable controls, respectively. ULCs consisted of 90% Poly(N-isopropylacrylamide) (NIPAm) and 10% Acrylic Acid (AAc) and 7% BIS particles consisted of 83% NIPAm, 10% AAc, and 7% N,N′-methylenebisacrylamide (BIS). A 100 mL precipitation synthesis of 140 mM monomer solutions and varying amounts of SDS (4 mM for ULCs, 2 mM SDS for 7% BIS) were filtered through a 0.2 μm filter with AAc added 10 minutes prior to the addition of 1 mM ammonium persulfate (APS). The mixture was then allowed to react for 6 hours at 70° C. flux with nitrogen purge. Resulting product solutions were then allowed to cool to room temperature and filtered against glass wool. Dialysis in 1000 kDa tubing against water was then conducted for 48 hours with 2 hour water changes for the latter 24 hours. The purified particle solutions were then frozen, lyophilized and resuspended at desired concentrations.

Dilutions of particle solutions were analyzed on a Malvern NanoSight NS300 for hydrodynamic radius characterization with size and distribution determined from 10 measurements of 30 seconds or 5 measurements of 60 sec. Particle size and morphology was also analyzed via atomic force microscopy (AFM) imaging. For AFM imaging, cover glass was cleaned by sonication in a sequence of solutions for 10 minutes per solution. The sequence of solutions is as follows: Alconox, deionized water, acetone, ethanol (200 proof), and isopropyl alcohol. 50 μL of particle suspension was then deposited onto cleaned cover glass and allowed to air-dry overnight. The cover glass is then mounted onto a microscope slide using epoxy. Air topography imaging was then conducted on a MFP 3D Bio AFM (Asylum). Topography and height trace images were then analyzed in ImageJ for particle diameter and height for at least 30 microgels per groups.

Particle morphology was analyzed by cryogenic scanning electron microscopy (cryoSEM) on a JEOL 7600F with Gatan Alto and Horiba CL Detector at 5 kV acceleration beam. 10 μL of particle solutions were deposited into the sample holder and plunged into nitrogen slush. Samples were then fractured and sputtered with gold.

Nanogel solutions were incubated with thrombin solutions for final thrombin concentrations of 50 U/ml or 75 U/ml and a final 0.5 mg/ml particle concentration for a total of 1 mL of solution. 100 μL time point aliquots were and analyzed for deformability, size and distribution, and morphology using AFM, Nanosight, and cryoSEM respectively. For AFM and Nanosight, aliquots were diluted 1:10 and analyzed as described in particle size characterization methods. For cryoSEM, a 1:4 dilution of TSNs in water to 100 U/ml thrombin was created with 50 μL timepoint aliquots collected for total volume 400 μL for a final particle concentration of ^{~}1.2 mg/mL. Timepoints were as follows for AFM and Nanosight: t=0, 2 hr, 4 hr, 24 hr, 48 hr, 96 hr, 1 wk. For cryoSEM, the timepoints were t=0, 2 hr, 6 hr, 24 hr, 48 hr, 96 hr, 1 wk.

To model the potential for thrombin to fully penetrate the thrombin-sensitive nanogels, nanogel solutions were lyophilized and then resuspended at 10 mg/ml in 5 μM 70 kDa FITC labeled dextran. 70 kDa dextran was selected as a model molecule, because 70 kDa is close to the molecular size of thrombin (72 kDa). After 24 hours, 10 μL of the suspension was deposited onto cleaned cover glass, allowed to dry, mounted to a microscope slide and then imaged on a LSM 880 confocal microscope. The resulting images were then analyzed by fluorescent trace on ImageJ. This procedure was also repeated on nanogels that have been exposed to thrombin for 24 hours and 48 hours.

Sheep anti-fibrin Fragment E polyclonal antibody was coupled to the AAc groups by EDC/NHS chemistry. PLPs refer to the products of the coupling of particles and Fragment E antibody. PLPs were purified by dialysis against H₂0 using 1,000 kDa dialysis tubing for 24 hours. The product was then lyophilized for 24-48 hours. PLP particles were then weighed and resuspended at 10 mg/ml. Products of anti-fibrin antibody coupling to particles are referred to as <particle type>-PLPs (e.g.: thrombin-sensitive nanogels conjugated to anti-fibrin Fragment E=thrombin-sensitive PLP).

Treatment clots were made of 2 mg/ml fibrin, 0.1 U/ml thrombin, 0.25 mg/ml of particles (TS-PLP, small ULC-PLP, small 7% BIS-PLP, or ULC-PLP) and 10% volume 10×HEPES buffer and allowed to polymerize for 48 hours. Control clots were created similarly without the presence of particles. All clots were made with total of 150 μL volume. 2 hours after initiation of polymerization, an overlay of 50 μL of 100 U/mL thrombin was added to clots. Clots were then imaged on a JEOL 7600F with Gatan Alto and Horiba CL Detector at 5 kV acceleration beam. 3 clots were imaged per group with at least three sites imaged per clot for analysis. Percent porosity for samples was determined using ImageJ.

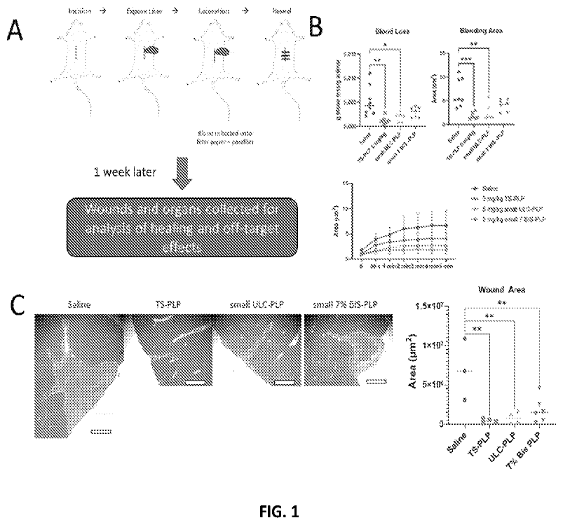

To evaluate in vivo hemostatic performance and wound healing outcomes, C57BL/6 mice were anesthetized with 2% isofluorane. All studies were conducted under approval by NCSU IACUC protocol. Treatments were administered by tail vein injection and allowed to circulate for 5 minutes. Treatment groups for this study were Saline, ULC-PLP, TS-PLP and 7% BIS-PLP. All PLP-based groups were administered intravenously at 100 μL of 5 mg PLPs/kg animal suspended in sterile saline. The liver of each mouse was then exposed. A liver laceration of 0.5 cm length was then made extending from the apex of the exposed left lobe (FIG. 1A) and blood was collected onto pre-weighed filter paper and parafilm. Bleeding over time was imaged at t=0, 30 s, 1 min, 2 min, 3 min, 4 min, and 5 min. At t=5 min, the filter paper and parafilm assembly was then measured. The liver was then returned to the abdomen and mice were aseptically sutured closed. Mice were then monitored daily for 1 week for body weight and signs of pain. After 1 week, mice were then sacrificed, and organs (wound site, healthy liver lobe, kidney, spleen, lungs, and heart) were harvested for histology. Blood loss was evaluated by determining the weight of blood collected onto the filter paper and parafilm assembly and normalizing the resulting mass by the respective animal's body weight. Filter paper area covered in blood (referred to as a bleeding area) was measured from each image taken during the bleeding assay using ImageJ. A separate study was conducted utilizing a terminal liver laceration model to determine particle presence immediately after injury. Injuries were performed on male C57/bl6 as described above and animals were sacrificed following the 5 minute bleeding period. Organs were harvested and prepared for histological analysis as described above.

Histological analysis was conducted with hematoxylin & eosin (H&E) staining and immunofluorescent labeling for platelets, fibrin, particle treatments, and cell nuclei. To label platelets, monoclonal anti-integrin beta 3/CD61 SJ19-09 antibody from Novus Biologicals was used as primary antibody with secondary Alexa 594 antibody. To label fibrin, monoclonal UC45 was used as primary antibody with secondary Alexa 594 antibody. To label particle treatments, a polyclonal anti-sheep 488 FITC-conjugated secondary antibody was used, tagging the fibrin specific antibody attached to particles. Cell nuclei were labeled by DAPI. Images were captured on an EVOS Auto FL microscope at 4× magnification for H&E stained wounds and 10× magnification for all other organs and fluorescently labeled wounds. For each fluorescently labeled wound, 3 images at different sites were taken.

To quantify wound healing outcomes, wound area and fluorescence thresholding area were measured on ImageJ. Wound area was measured from H&E stained sections. Total areas for fibrin, particle presence, and CD61+ expression were measured by conducting particle analysis on 8-bit images.

To quantify off-target effects and clotting, peripheral organ (healthy liver lobe, heart, lung, kidney, spleen) sections were fluorescently labeled for fibrin and particles using the same methods described for fluorescent labeling for wounds. Fibrin and particle presence were also quantified using the same methods.

All statistical analysis was conducted in GraphPad Prism 9. Bleeding area vs. time data was analyzed by two-way ANOVA. All other data was analyzed by one-way ANOVA with Tukey's post hoc test for multiple comparisons.

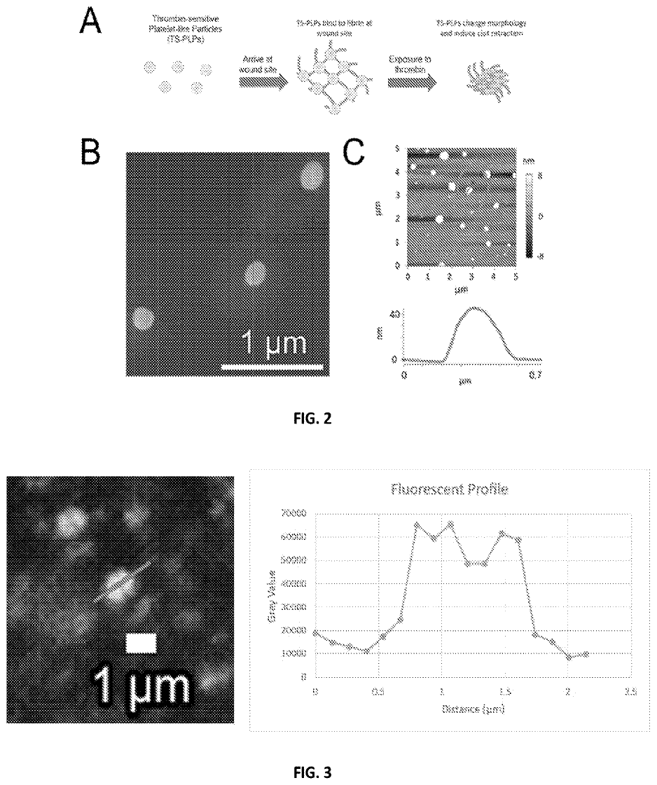

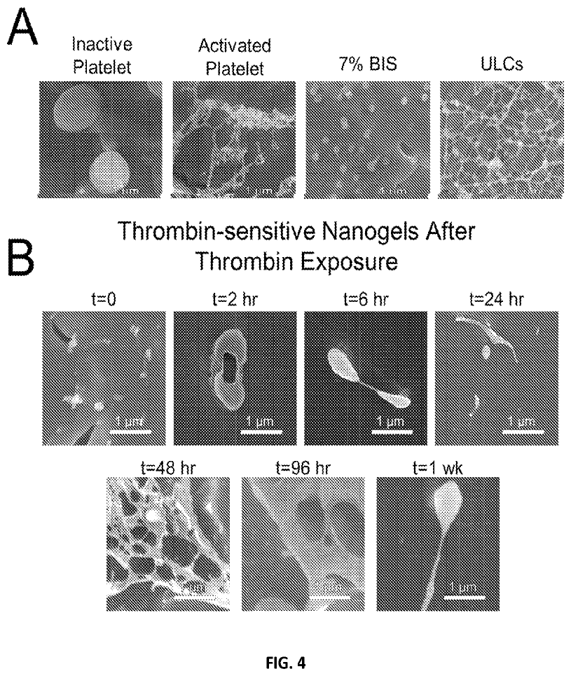

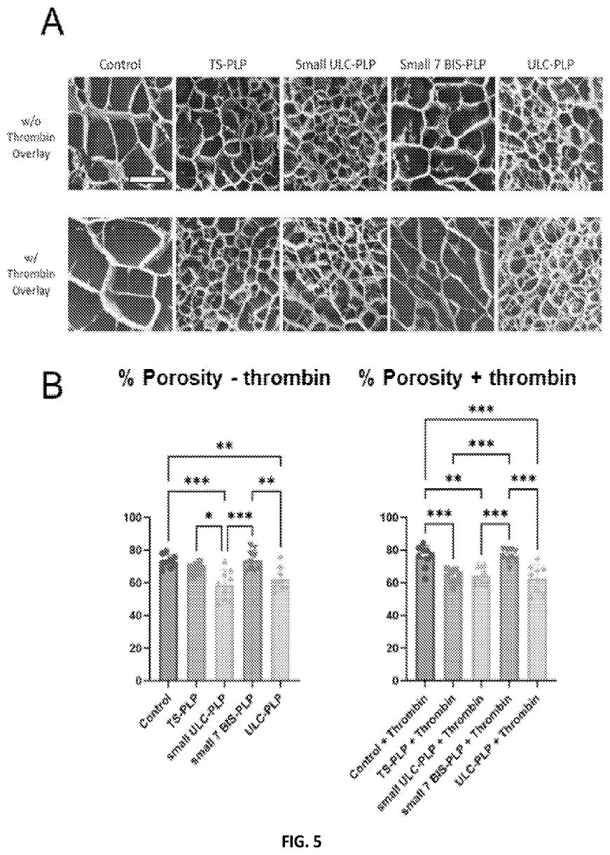

Unmodified thrombin-sensitive nanogels were determined to have average dry diameter of 330±50 nm and average dry height of 33±10 nm. When imaged for morphology on cryoSEM, the thrombin-sensitive nanogels displayed a rounded morphology (FIG. 2). When exposed to 50 U/ml or 75 U/mL thrombin, population distribution markers on Nanosight show significantly decreased size starting from t=2 hr. However, size increased after 24 hours for samples exposed to 75 U/ml thrombin, potentially due to decreased crosslinks leading to increased swelling. AFM data with 50 U/mL thrombin shows no significant changes in particle diameter until t=4 hr and 96 hr. In the presence of 50 U/ml thrombin, particle height also significantly decreased starting from t=2 hr (FIG. 3). Size increases at later time points, in combination with decreasing particle height, may indicate swelling and increased deformability. This change in particle behavior was further confirmed with imaging of aliquots on cryoSEM for morphology. At later time points, the thrombin-sensitive nanogels displayed more stellate-like morphology, much like active platelet morphology and ULC microgels (FIG. 4).

Thrombin degradation assays showed that particles did not fully degrade. Thus, loading behavior was investigated to determine if thrombin was successfully penetrating the entirety of the particles. Fluorescent levels were observed to be higher at the edges of nanogels and lower in the center (Supplemental FIG. 2).

After confirming that the nanogels change deformability in the presence of thrombin, we then sought to evaluate if this was sufficient to induce clot retraction when the particles are coupled to a fibrin-binding antibody. Clots including no particles (control), TS-PLPs, size-matched ULC-PLPs (small ULC-PLPs), size-matched 7% BIS-PLPs (small 7 BIS-PLPs) and ULC-PLPs (corresponding to the size of our original design) were created, with +thrombin counterparts receiving a 100 U/mL thrombin overlay 2 hours after initiation of polymerization. All clots were then imaged on cryoSEM for clot structure (FIG. 5). The resulting images were then analyzed for % porosity, which is defined as black pixel area/total image pixels.

For clots without a thrombin overlay, average % porosity was determined to be 73±4 for control, 68±3 for TS-PLPs, 59±9 for small ULC-PLPs, 74±6 for small 7 BIS-PLPs, and 62±7 for ULC-PLPs. For clots with a thrombin overlay, average % porosity was determined to be 76±7 for control, 63±4 for TS-PLPs, 64±5 for small ULC-PLPs, 76±4 for small 7 BIS-PLPs, and 63±8 for ULC-PLPs. Without the addition of a thrombin overlay, it was found that clots with unmodified TS-PLPs incorporated do not have significantly lower % porosity whereas clots with small ULC-PLPs (p≤0.001) and ULC-PLPs have significantly lower % porosity (p≤0.01). Thus, the ULC-PLP particle types perform as expected. With the addition of a thrombin overlay, in contrast, clots with TS-PLPs demonstrate a significantly lower % porosity (p≤0.001), on par with lower % porosity observed in clots with small ULC-PLPs (p≤0.001) and ULC-PLPs (p≤0.001).

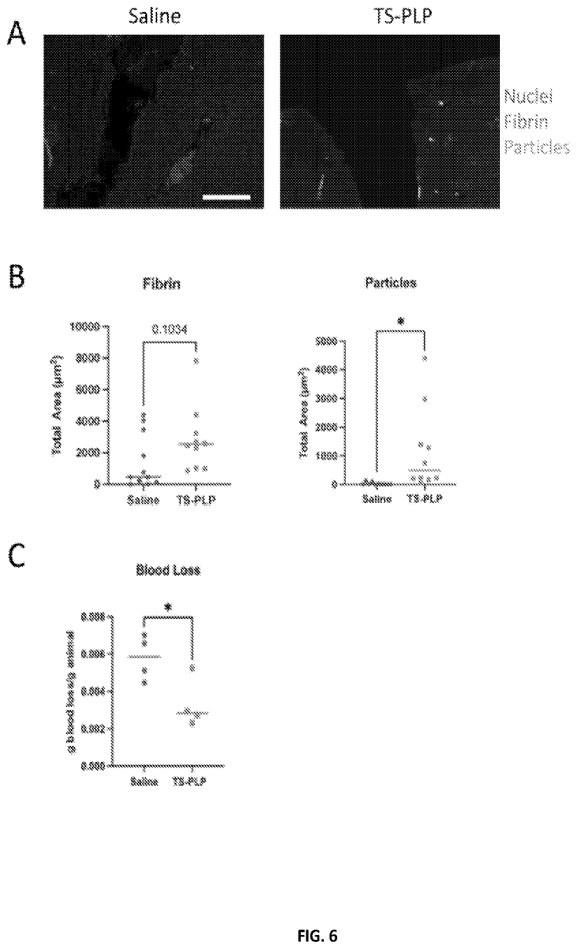

We next evaluated the hemostatic ability of the TS-PLPs in a survival traumatic liver laceration model. Average blood loss (g blood loss/g animal) was 5.2×10⁻³±3.1×10⁻³ for saline treated animals, 1.3×10⁻³±0.78×10⁻³ for TS-PLP treated animals, 2.0×10⁻³±0.96×10⁻³ for size-matched ULC-PLP treated animals, and 2.8×10⁻³±1.1×10⁻³ for size-matched 7% BIS-PLP treated animals. Average bleeding area was 6.7±2.9 cm² for saline treated animals, 1.8±0.7 cm² for TS-PLP treated animals, 2.7±1.8 cm² for size-matched ULC-PLP treated animals, and 4.0±1.2 cm² for size-matched 7% BIS-PLP treated animals. In mice treated with TS-PLPs, significantly lower blood loss and bleeding area were observed compared to saline treated animals. Additionally, TS-PLPs reduce bleeding to a similar extent as ULC-PLPs. (FIG. 1) Similarly, in an additional cohort of animals in terminal studies, TS-PLPs significantly reduced average blood loss compared to saline controls. (FIG. 6).

To evaluate longer term healing outcomes after cessation of bleeding, wound area was measured from H&E-stained sections, fibrin positive area, CD61+ area, and particle presence were measured from immunofluorescent stained sections collected from animals 7 days post-injury. Average wound area was 6.9×10⁶±3.9×10⁶ μm² for saline treated animals, 0.5×10⁶±0.3×10⁶ μm² for TS-PLP treated animals, 0.8×10⁶±0.7×10⁶ μm² for size-matched ULC-PLP treated animals, and 1.8×10⁶±1.6×10⁶ μm² for size-matched 7% BIS-PLP treated animals. Average fibrin area was 4585±1417 μm² for saline treated animals, 16458±11202 μm² for TS-PLP treated animals, 3725±2058 μm² for size-matched ULC-PLP treated animals, and 3414±3140 μm² for size-matched 7% BIS-PLP treated animals. Average CD61+ area was 4762±3353 μm² for saline treated animals, 2545±2585 μm² for TS-PLP treated animals, 3945±1739 μm² for size-matched ULC-PLP treated animals, and 5668±5305 μm² for size-matched 7% BIS-PLP treated animals. Average particle presence area was 0 μm² for all treatment groups. In terminal, acute studies, the average fibrin area was 1433±1716 μm² for saline treated animals and 2849±2073 μm² for TS-PLP treated mice. The average particle presence area in these animals was 35.6±47.41 μm² for saline treated animals and 1175±1449 μm² in TS-PLP treated mice.

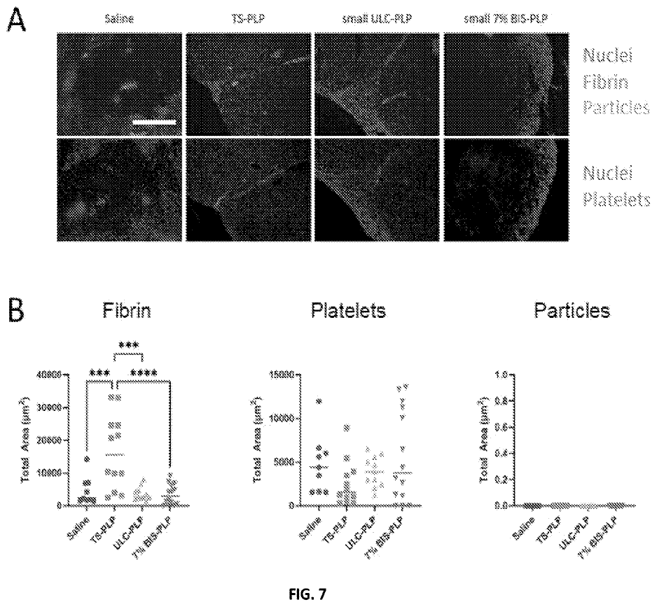

Overall, significantly smaller wound areas and CD61+ expression were observed one week after injury in animals treated with TS-PLPs, size-matched ULC-PLPs, and size-matched 7% BIS-PLPs. However, fibrin presence was significantly higher only in animals treated with TS-PLPs. The higher presence of fibrin one week after injury indicates that fibrin-stability is greater in vivo in these animals. Additionally, no significant differences were observed in particle presence across all groups, indicating that any particles administered to mice were no longer retained in the injury site 1 week post-injury (FIG. 7). However, histological analysis of wounds harvested immediately after the bleeding period revealed greater fibrin signal and significantly increased particle signal, indicating TS-PLPs are wound targeting (FIG. 6).

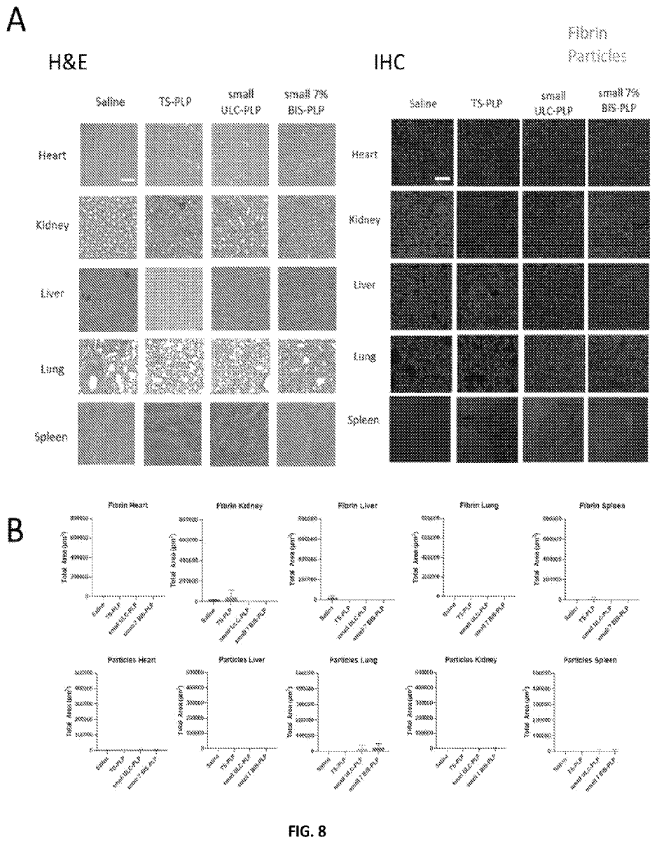

To evaluate potential off-target effects, peripheral organs were stained by H&E for tissue morphology and fluorescently labeled for fibrin and particles and measured for fibrin-positive and particle presence areas. Across all organs, tissue morphology appeared normal and no significant differences in fibrin and particle presence were detected between treatment groups (FIG. 8).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. Thrombin-sensitive platelet-like particles, wherein the particles comprise a thrombin-degradable crosslinked poly(meth)acrylamide.

2. (canceled)

3. The particles of claim 1, wherein the particles comprise core-shell particles; and
wherein the core and/or the shell comprises the thrombin-degradable crosslinked poly(meth)acrylamide; or
wherein the core comprises the thrombin-degradable crosslinked poly(meth)acrylamide, and the shell comprises poly(meth)acrylamide crosslinked with a non-thrombin degradable crosslinker.

4-10. (canceled)

11. The particles of claim 1, wherein the particles comprise a thrombin-degradable crosslinker comprising a five-reside amino acid fragment comprising the formula:
-Pro-Arg-X1-X2-Arg-[[,]]
wherein:
X¹ is Ser, Ala, Gly, or Thr; and
X² is a non-acidic amino acid.

12. The particles of claim 1, wherein the particle comprise a thrombin degradable crosslinker comprising the formula:
Leu-Val-Pro-Arg-Gly-Ser;
Gly-Gly-Leu-Val-Pro-Arg-Gly-Ser-Gly-Ala Ser;
Ala-Lys-Gly-Ala-Leu-Val-Pro-Arg-Gly-Ser-Ala-Gly-
Lys;
or
Gly-dPHe-Pro-Arg-Gly-Phe-Pro-Ala-Gly-Gly-Lys.

13-40. (canceled)

41. The particles of claim 1, further comprising a fibrin-binding moiety comprising at least one fibrin-binding IgG antibody, fibrin-binding peptide, Fragment D binding antibody, or antibody fragment that binds fibrin.

42-45. (canceled)

46. The particles of claim 1, further comprising a small molecule therapeutic agent and/or a biologic therapeutic agent.

47-49. (canceled)

50. The particles of claim 1, wherein the particles have an average dry diameter no greater than 1,000 nm, an average dry height no greater than 50 nm, and/or a polydispersity index no greater than 0.4; and
wherein the polydispersity index is calculated from the square of the standard deviation divided by the mean particle diameter determined from Nanosight analysis.

51. (canceled)

52. A powder, nanogel, or wound dressing comprising the particles of claim 1.

53-56. (canceled)

57. A method of making the particles of claim 1, the method comprising polymerizing a mixture comprising (meth)acrylamide and a thrombin-degradable crosslinker.

58. The method of claim 57, wherein the monomer mixture further comprises (meth)acrylic acid and/or acrylic acid.

59-65. (canceled)

66. The method of claim 58, wherein the monomer mixture comprises N-isopropylacrylamide, acrylic acid, and a thrombin-degradable crosslinker.

67-71. (canceled)

72. The method of claim 57, wherein the monomer mixture is combined with an initiator.

73-74. (canceled)

75. The method of claim 72, wherein the initiator comprises ammonium persulfate and tetramethylethylenediamine.

76-79. (canceled)

80. The method of claim 72, wherein the (meth)acrylamide and thrombin-degradable crosslinker are stirred for a period of at least 10 minutes prior to the addition of the initiator.

81-83. (canceled)

84. The method of claim 57, further comprising:
separating the thrombin-degradable crosslinked poly(meth)acrylamide from the mixture;
combining the separated thrombin-degradable crosslinked poly(meth)acrylamide in a second monomer mixture comprising (meth)acrylamide; and
polymerizing the second monomer mixture to give a core/shell particle.

85-86. (canceled)

87. The method of claim 84, wherein the second monomer mixture comprises a thrombin-degradable crosslinker and/or a non-thrombin-degradable crosslinker.

88-90. (canceled)

91. The method of claim 87, wherein the non-thrombin-degradable crosslinker comprises N,N′-methylenebis(acrylamide), N,N-(1,2-dihydroxyethylene)bisacrylamide, ethylene glycol diacrylate, di(ethylene gycol) diacrylate, tetra(ethylene glycol) diacrylate, ethylene glycol dimethacrylate, di(ethylene glycol) dimethacrylate, tri(ethylene glycol) dimethacrylate, or a combination thereof.

92-95. (canceled)

96. The method of claim 84, further comprising conjugating a fibrin-binding moiety to the core and/or the shell.

97. (canceled)

98. A method of promoting wound healing in a subject in need thereof, the method comprising contacting a wound with the particles of claim 1.

99-109. (canceled)

110. A method of treating cancer in a patient in need thereof, the method comprising administering to the patent the particles of claim 1, wherein the particles further comprise a chemotherapeutic agent.
